(19) United States
(12) Patent Application Publication
(10) Pub. No.: US 2025/0121113 A1
(43) Pub. Date: Apr. 17, 2025

(54) PORTABLE AND FILTER-FREE SINGLE-PASS AIR DISINFECTION SYSTEM FOR DISINFECTION OF CONTAMINATED AIRFLOW OR BIOAEROSOLS

(71) Applicant: City University of Hong Kong, Hong Kong (HK)

(72) Inventors: Chi Keung LAI, Hong Kong (HK); Huihui ZHANG, Xin Yu City (CN); Minghao WANG, Hong Kong (HK); Yuhao LU, Zhang Zhou City (CN)

(21) Appl. No.: 18/806,743

(22) Filed: Aug. 16, 2024

Related U.S. Application Data
(60) Provisional application No. 63/590,000, filed on Oct. 13, 2023.

(51) Int. Cl. A61L 9/20

(52) U.S. Cl. CPC A61L 9/20; A61L 2209/12; A61L 2209/20

(57) ABSTRACT

Two portable disinfection systems for indoor air disinfection are provided. One system is designed to be equipped with an external Far-UVC lamp as UV source, while the other is designed to be equipped with an internal Far-UVC tubular lamp as UV source. The system's portability makes it versatile and applicable in many different scenarios. The portability of these systems makes them versatile and suitable for various scenarios, including room disinfection and public transportation. They can function as standalone air disinfection units or be integrated with existing technologies to further reduce airborne pathogen concentrations.

### CROSS-REFERENCE TO RELATED APPLICATIONS

The present application claims the priorities from the U.S. provisional patent application Ser. No. 63/590,000 filed Oct. 13, 2023, and the disclosure of which is incorporated herein by reference in its entirety.

### FIELD OF THE INVENTION

The present invention generally relates to devices related to ultraviolet (UV) radiation, including sterilization technologies, air purification systems, and UV radiation applications.

### BACKGROUND OF THE INVENTION

In response to the challenges posed by airborne transmission of pathogens like SARS-CoV-2, various technologies have been explored to enhance indoor air safety. Traditional approaches in commercial buildings, such as mechanical ventilation and filtration systems, have limitations including high costs and maintenance issues. Mechanical ventilation, while effective, can be impractical to implement in existing structures, and filters, while capable of capturing bioaerosols, require frequent replacement and can become breeding grounds for pathogens if not maintained properly.

Ultraviolet germicidal irradiation (UVGI) emerges as a promising alternative. UVGI directly deactivates microbes, significantly reducing indoor microbial concentrations. Integrated with High-Efficiency Particulate Air (HEPA) filters, UVGI systems have demonstrated substantial reductions in airborne bacteria and viral infectivity in commercial settings. Studies indicate up to a 73% reduction in airborne bacteria when UVGI is combined with HEPA filtration³. In another study, products equipped with 254 nm UVC and HEPA (High-efficiency particulate air Filter) filters were reported to reduce the viral infectivity of indoor Phi6-bacteriophages⁴. Even in the absence of HEPA filters, these products still achieved over 70% reduction in viral infectivity.

Types of UVGI systems include: (1) Upper-room UVGI: positioned high in rooms, this system disinfects air in upper regions directly. However, its efficacy can be influenced by local airflow patterns; (2) In-duct UVGI: integrated into central HVAC systems, in-duct UVGI installations disinfect air as it circulates through the building's ventilation network; (3) Portable UVGI systems: these have gained popularity during the pandemic for their effectiveness in mitigating airborne and surface-borne pathogen transmission. Combining medium efficiency filters with UVGI, portable systems have shown significant pathogen reduction without the need for filter replacements.

An UVGI equipped with 254 nm UVC was reported to reduce the virulence of indoor Phi6-bacteriophages to 28.7% while the value was further reduced to 0.6% when the HEPA filter was added⁵. Thus, the UVGI is the potential to replace the filter to purify the indoor air when the particulate matters (PM) are not the main concern. However, the conventional use of low-pressure mercury lamps emitting 254 nm UV raises environmental and health concerns due to mercury content and potential skin and eye hazards. On the other hand, high flow rates can pose challenges to the disinfection performance of the system.

A prototype study conducted at a flow rate of 1254 L/min demonstrated that the device can perform effective disinfection by applying UV reflective materials and employing appropriate structural design⁶. Nevertheless, the study mentioned the presence of high surface temperatures in the air channel, leading to a concern regarding heat dissipation. This was attributed to the installation of many UV sources within the air channel.

Due to the limitations associated with low-pressure mercury lamps, other wavelengths from the UV regime and materials have been explored and tested. For example, Deep-Ultraviolet emitted by LEDs (light-emitting diodes), 405 nm blue light. The antimicrobial effects of UVC-LED have been demonstrated by several studies⁷⁻⁹. However, challenges remain in terms of the low output of individual LED and significant heat dissipation that occurs during LED illumination. The use of 405 nm blue light, while viable, exhibits relatively low efficacy in microbial inactivation and often necessitates high doses to achieve effective results.

The field of air disinfection technology faces numerous challenges that require continued in-depth research and innovation to enhance efficiency, reduce costs, and improve safety and health aspects, thereby better meeting the diverse needs across different environments.

### SUMMARY OF THE INVENTION

The present invention aims to evaluate portable systems that utilize various ultraviolet light sources with unique features to achieve high disinfection efficacy at realistic high airflow rates. This system efficiently inactivates microbes using Far-UVC light at high airflow rates. Unlike existing filter-based air purifiers, the designed system of the present invention eliminates airborne microbes from indoor air by inactivating them, thereby completely eradicating the risk of secondary transmission.

The present invention implements two innovative measures to enhance the disinfection efficacy of the system: optimizing exposure flow and increasing irradiation intensity. By redirecting exposure pathways and improving internal reflection, the disinfection system achieves significantly improved efficacy.

In a first aspect, the present invention provides a portable and filter-free single-pass air disinfection system for disinfection of contaminated airflow or bioaerosols. The portable and filter-free single-pass air disinfection system includes a disinfection chamber and one or more exhaust fans equipped on one side of the disinfection chamber to facilitate airflow through the disinfection chamber. The disinfection chamber includes a premixing zone, a treatment zone and a sampling zone. The premixing zone, the treatment zone and the sampling zone are interconnected. There is at least one partition positioned between the premixing zone and the treatment zone. The premixing zone includes at least one air inlet for introducing the contaminated airflow or the bioaerosols into the disinfection chamber. The treatment zone includes an ultraviolet (UV) irradiation source configured to irradiate the contaminated airflow or the bioaerosols within the disinfection chamber. The sampling zone includes a liquid impinger connected to a vacuum pump for air sampling, and at least one vent outlet for cleaned air.

In one embodiment, the partition is made of UV-penetrable materials, and its length is shorter than the width of the disinfection chamber.

In one embodiment, the UV irradiation source is a Far-UVC source emits light at a wavelength of approximately 222 nm, the Far-UVC source includes a tubular lamp or a rectangular lamp.

In one embodiment, the rectangular lamp is positioned externally to the disinfection chamber.

In another embodiment, the tubular lamp is positioned internally within the treatment part.

In one embodiment, the at least one partition re-directs the contaminated airflow or the bioaerosols to extend an airflow pathway to a region with high UV irradiation.

In one embodiment, the least one partition and internal walls of the disinfection chamber are covered with reflected materials with a reflectivity of at least 90% to enhance UV reflectance, the reflected materials comprise aluminum foil.

In one embodiment, the portable and filter-free single-pass air disinfection system has an airflow rate of at least 100 m³/h.

In one embodiment, the portable and filter-free single-pass air disinfection system inactivates pathogens in the contaminated airflow or bioaerosols. The portable and filter-free single-pass air disinfection system achieves an inactivation rate of at least 80% for Salmonella enterica, at least 15% for Staphylococcus epidermidis, and at least 20% for MS2 bacteriophage.

In another embodiment, the portable and filter-free single-pass air disinfection system includes an input terminal connected to a current input device for controlling current of the one or more exhaust fans.

In one embodiment, the disinfection chamber is made of at least one acrylic plate with a thickness of 1 mm to 10 mm.

In a second aspect, the present invention provides a method for disinfection of contaminated airflow or bioaerosols, including: preparing a filter-free disinfection chamber having a premixing zone, a treatment zone and a sampling zone; introducing the contaminated airflow or the bioaerosols into the premixing zone; directing the contaminated airflow or the bioaerosols towards the treatment zone; and collecting and analyzing irradiated air samples to determine microbial concentration. There is at least one partition inserted between the premixing zone and the treatment zone. The treatment zone includes an ultraviolet (UV) irradiation source configured to irradiate the contaminated airflow or the bioaerosols within the disinfection chamber, wherein the UV irradiation source comprises Far-UVC source emits light at a wavelength of approximately 222 nm.

In one embodiment, one or more exhaust fans are equipped on one side of the disinfection chamber to facilitate airflow through the disinfection chamber.

In one embodiment, the Far-UVC source comprises a tubular lamp or a rectangular lamp. The rectangular lamp is positioned externally to the disinfection chamber. The tubular lamp is positioned internally within the treatment zone.

In one embodiment, the method further includes covering the at least one partition and internal walls of the disinfection chamber with reflected materials with a reflectivity of at least 90% to enhance UV reflectance.

In another embodiment, the method further includes pre-disinfecting the disinfection chamber with an UV lamp before introduction of the contaminated airflow or the bioaerosols.

In yet another embodiment, the method further includes installing a safety mechanism to automatically shut off the UV irradiation source if the disinfection chamber is opened during operation.

In yet another embodiment, the method further includes calibrating the UV irradiation source to ensure consistent irradiance levels across the disinfection chamber.

The invention has the following advantages:

The inactivation efficacy of the portable and filter-free single-pass disinfection systems has been significantly improved. By inserting partitions to redirect airflow and enhancing UV reflectance, both the Internal-Tube-Equipped (ITE) system and the External-Module-Equipped (EME) system achieved substantial gains in efficacy without the need for additional UV sources. With two original modifications, the EME system reached inactivation efficacies of 93.4% for S. enterica, 51.8% for S. epidermidis, and 52.5% for MS2. Similarly, the ITE system attained inactivation efficacies of 97.6%, 54.4%, and 55.1% for the same microorganisms, respectively. Both system configurations demonstrated a high level of inactivation efficacy, with overall enhancements of at least 200%.

Compared to upper-room UVGI installations, the portable and filter-free single-pass disinfection systems present a 23.0% to 29.2% higher equivalent ACH of removing Far-UVC resistant microorganisms. This demonstrates the potential of using this system in small spaces, such as residential houses and private offices.

### BRIEF DESCRIPTION OF THE DRAWINGS

Embodiments of the invention are described in more details hereinafter with reference to the drawings, in which:

FIG. 1 shows the layout of the disinfection box chamber. (Unit: mm). a=bioaerosols inlet, b₁₋₃=air inlets, c₁₋₂=sampling points, d₁₋₂=exhaust fans;

FIG. 2A shows configuration of baseline EME disinfection system. FIG. 2B shows configuration of baseline ITE disinfection system;

FIG. 3A shows spectral distribution emitted by the rectangular lamp module. FIG. 3B shows spectral distribution emitted by the tubular lamp. FIG. 3C shows the ozone concentration inside the box chamber with respect to the operation time of the lamps;

FIG. 4A shows the diagram of the modified EME disinfection systems with bare partitions. FIG. 4B shows the diagram of the modified ITE disinfection systems with bare partitions;

FIG. 5A shows a top view of the modified EME disinfection systems with aluminum (Al) foil cover partitions. FIG. 5B shows a top view of the modified ITE disinfection systems with Al foil cover partitions;

FIG. 6A shows UV irradiance distribution measured inside the baseline EME system. FIG. 6B shows UV irradiance distribution measured inside the baseline ITE system. For each grid point, there are two numeric, the top indicates the irradiance measured at Z=75 while the bottom indicates the irradiance measured at Z=25 (Unit: μW/cm² for UV irradiance; mm for dimension);

FIGS. 7A-7B show measured UV intensity distribution in a UV treatment section of modified EME and ITE system. FIG. 7A indicates bare partitions while FIG. 7B indicates Al foil covered partitions;

FIG. 8 shows flow rates of the baseline and modified EME/ITE systems. (“V” means p-value<0.05 while “x” means p-value>0.05. The brackets indicate ITE system); and

FIG. 9A shows inactivation efficacies of the baseline and enhanced EME/ITE system against S. enterica. FIG. 9B shows inactivation efficacies of the baseline and enhanced EME/ITE system against S. epidermidis. FIG. 9C shows inactivation efficacies of the baseline and enhanced EME/ITE system against MS2.

### DETAILED DESCRIPTION

In the following description, portable and filter-free single-pass air disinfection systems utilizing different form factors of Far-UVC sources are set forth as preferred examples. It will be apparent to those skilled in the art that modifications, including additions and/or substitutions may be made without departing from the scope and spirit of the invention. Specific details may be omitted so as not to obscure the invention; however, the disclosure is written to enable one skilled in the art to practice the teachings herein without undue experimentation.

Conventional air purifiers use filtration to remove particulate matter and bioacrosols from indoor air, making them a popular choice for their portability and reliable performance. However, viruses can be as small as tens of nanometers, requiring the use of HEPA (high-efficiency particulate air) filters to capture them effectively. However, there are several downsides to using HEPA filters. The HEPA filters cannot be cleaned and reused. To maintain their expected filtration performance, HEPA filters must be replaced regularly. Furthermore, HEPA filters are expensive and considered a one-off consumable, making filtration-based air purifiers a non-sustainable product.

There are two possible arrangements for the UV source in the single-pass air disinfection system. The first is an external arrangement, where the lamp is positioned outside the system. In this setup, the irradiation passes through openings or transparent windows to disinfect the airflow. The second arrangement is an internal one, where the lamp, usually a bare tube, is securely installed within the air channel. The internal tube provides wider UV coverage than the external illuminating surface. On the other hand, an external UV source allows for heat dissipation outside of the system without affecting the airflow inside. Accordingly, in the present invention, two air disinfection systems are provided using different configurations of Far-UVC sources to disinfect indoor air. The disinfection systems are portable, efficient, environmentally friendly, and has low operating cost and maintenance costs.

Two types of light sources:

The first system, referred to as the External-Module-Equipped (EME) system, includes a rectangular UV source module mounted on the outer surface of the disinfection box chamber. In the baseline EME system, air flows directly through the UV treatment section, resulting in a low dose of UV exposure. Moving the rectangular lamp to the opposite side could increase the air's exposure to UV irradiation. However, because the airflow is perpendicular to the direction of the UV light, the exposure range would be limited by the lamp's narrow irradiation angle.

The second system, named the Internal-Tube-Equipped (ITE) system, integrates a UV tube centrally within the box chamber.

Additionally, extra partitions can be inserted to redirect the contaminated air towards areas with high UV irradiance. This also aims to extend the exposure time by increasing air resistance.

In one embodiment, the light source is an optical filtered 222 nm Far-UVC tube, which is mercury free.

Regarding Far-UVC sources, 222 nm Far-UVC emerges as a sustainable option with advanced emission source technology and proven antimicrobial efficacy. Moreover, 222 nm Far-UVC is considered safer than 254 nm UVC due to limited skin penetration and minimal DNA absorption. The 222 nm UV source has a longer lifespan than the HEPA filter. The recommended replacement period of the air filter is 4 to 6 months, while the expected lifespan of the 222 nm UV lamp is over 4000 hours (16 months for 8 hours used daily). Besides, the expense of 222 nm UV lamps is reducing due to the technology developing and manufacturing expansion.

To assess air disinfection efficacy of the portable disinfection system, two bacteria, Salmonella enterica and Staphylococcus epidermidis, and one bacteriophage, MS2 are selected as challenges for the systems operating at a flow rate over 100 m³/h.

The baseline EME system inactivates the microbes with the efficacies of 81.4%, 16.9%, and 25.1% for S. enterica, S. epidermidis, and MS2, respectively. In comparison, the baseline ITE system demonstrates higher inactivation efficacies due to a wider irradiated area, with respective efficacies of 94.4%, 25.8%, and 37.8% for the tested microorganisms.

The air disinfection systems include a few original features improving the exposure of pathogens and UV light intensity that can significantly enhance the disinfection efficacy of the system. Two enhancement measures are implemented by redirecting airflow to the areas with high UV irradiance and enhancing UV reflectance, increasing the inactivation efficacy of the systems by at least 200%. When tested against Staphylococcus epidermidis, compared with an upper-room Far-UVC, the modified EME and ITE systems present an increase in inactivation rate by 23.0% and 29.2% respectively. These results demonstrate the potential of the portable disinfection systems to efficiently and sustainably inactivate a broad spectrum of microbes.

In addition to Salmonella enterica, the portable disinfection system of the present invention may inactivate other Gram-negative bacteria, including but is not limited to Escherichia coli, Pseudomonas aeruginosa, Klebsiella pneumoniae, Neisseria gonorrhoeae, Haemophilus influenzae, Helicobacter pylori, Proteus mirabilis, Vibrio cholerae, Acinetobacter baumannii.

In addition to Staphylococcus epidermidis, the portable disinfection system of the present invention may inactivate other Gram-positive bacteria, including but is not limited to Staphylococcus aureus, Streptococcus pyogenes, Streptococcus pneumoniae, Enterococcus faecalis, Bacillus anthracis, Listeria monocytogenes, Corynebacterium diphtheriae.

Moreover, the application of aluminum (Al) foil further improves the inactivation efficacy of the systems by providing UV reflection. These modifications allow the EME system to achieve comparable, or even superior, inactivation efficacy compared to the ITE system, while maintaining the same power consumption of the UV source.

### EXAMPLE

### Example 1—Materials and Methods

### Tested Microorganism

In this example, Salmonella enterica (S. enterica) (ATCC 53648), Staphylococcus epidermidis (S. epidermidis) (ATCC 12228) and MS2 were selected as the tested microbes. S. enterica and S. epidermidis are both commonly found in the indoor environment and widely used in disinfection experiments. Bacteriophages MS2, a non-enveloped and single-stranded RNA virus, was tested as a surrogate for SARS-CoV-2 and influenza.

### Preparation of the Tested Microorganism

The colony of each strain, grown on the Nutrient Broth (NB; Difco) agar first, was inoculated into NB and incubated at 37° C. for 24 hours to get a stationary growth phase. The bacteria were harvested by centrifugation at 6000 rpm (revolution per minute) and suspended in 50 mL of 0.9% saline with a concentration of 10⁸ to 10⁹ CFUs (colony-forming units)/mL for the subsequent experiment.

MS2 was cultured by a double-agar layer approach. Escherichia. coli (E. Coli, ATCC 15597), the host of MS2, was first grown in TSB (Tryptone Soya Broth, Difco) at 37° C. for 1 hour to reach the early log phase. Following that, 1 mL of E. Coli overnight culture was mixed with 0.05 mL of stock MS2 resuspension in 5 ml fresh TSB. Then, the bacterial-phage mixture was added into melted 45° C., 5 mL, 0.7% (wt/vol) fresh soft TSA (Tryptone Soya agar, Difco) for vortex mixing. After vortex mixing, the mixture was poured onto a 1.5% solid TSA plate and left to solidify for 3 to 5 minutes. Subsequently, the plate was incubated at 37° C. for 24 hours. The soft agar on the overnight plates was scraped out with a disposable spreader and resuspended in 25 mL of 0.9% saline. The suspension was centrifuged at 6000 rpm for 15 min to remove cells and debris. The supernatant was filtered through 0.22 μm polyethersulfone (PES, Millipore) membranes and kept at 4° C. until further investigation. The final concentration of MS2 solution was 10¹¹ to 10¹² plaque-forming units (PFUs)/mL.

### Example 2—Preparation of Portable and Filter-Free Single-Pass Disinfection Systems

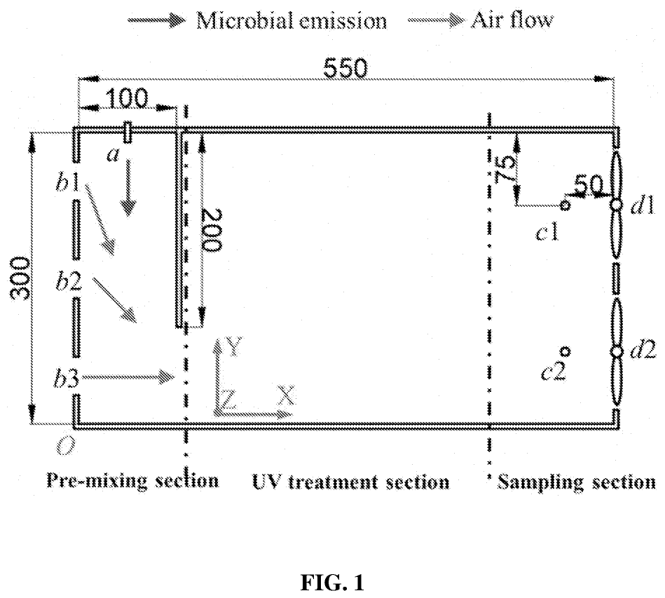

All systems included three essential hardware components: a disinfection box chamber, a UV source, and two exhaust fans. The box chamber, with dimensions of 550 mm×300 mm×150 mm (X, Y, Z), was made of 5 mm thickness acrylic plates, which were transparent but have virtually zero Far-UVC light penetration. The longitudinal section of the box chamber was depicted in FIG. 1, along with the detailed dimensions. The box chamber was divided into three sections: a pre-mixing section (X ranges from 0 to 100 mm), a UV treatment section (X ranges from 100 mm to 400 mm), and a sampling section (X ranges from 400 mm to 550 mm).

Referring to FIG. 1, the microorganisms were nebulized and injected into the box chamber from position a. Simultaneously, the indoor air was drawn into the box chamber through three circular openings b₁₋₃, each with a diameter of 40 mm. Inside the box chamber, the air and the microorganisms were mixed in the pre-mixing section, which was formed by partitioning the box chamber using an acrylic plate, and then entered the UV treatment section. While the aerosolized microbes were effectively mixed with the airflow during its passage through these sections, their distribution within the sampling section may not have been uniform. Given that there were two outlets for the airflow leaving the box chamber, air samples were individually collected from c₁ and c₂, which were near the outlets. These samples were combined to determine the concentration of microorganisms present within the system. Two axial fans (Delta, AFC1212D) were installed at positions d₁, d₂ to draw the air flow through the box chamber. The rated airflow rate of each fan is 148CFM (248.64 m³/h).

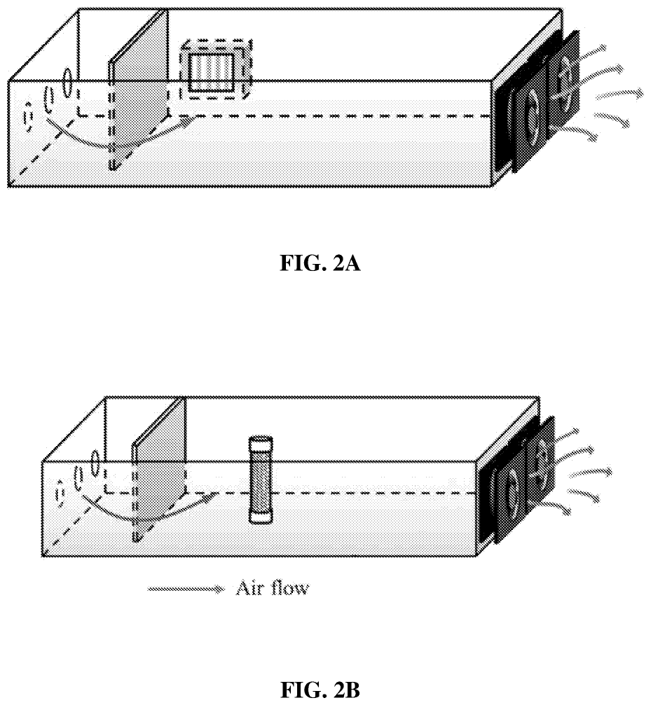

In this example, two distinct krypton-chloride Far-UVC sources were employed individually in the UV treatment section. The first source was a rectangular lamp module (15 W, Care 222, Ushio, Japan), while the second source was a tubular lamp (20 W, First UVC, China). These lamps were equipped with an optical window that filtered out all other emission wavelengths except for 222 nm. FIGS. 2A-2B illustrated the schematics of the two systems in their baseline configurations, which served as benchmarks for comparison.

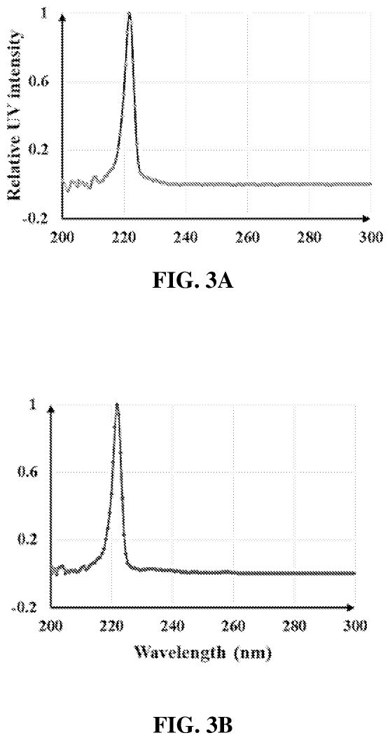

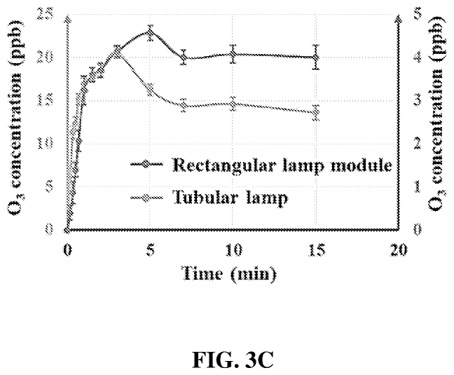

Spectral distribution and O₃ generation of the two lamps were measured and documented in FIG. 3A-3C. Both lamps had a peak emission at 222 nm with a full-width half maximum (FWHM) of 4 nm. The concentration of generated O₃ was measured by an O₃ monitor (Model 205, 2B Technologies) inside the box chamber near UV source under no airflow condition. Both lamps generated minimal O₃ during illuminating, with concentrations several times lower than the U.S. Environmental Protection Agency standard of 70 ppb34. Additionally, the measured concentrations were significantly lower than the concentration of 2300 ppb at which O₃ would exhibit antibacterial properties and cause extensive cell death35. It should also be noted that during the experiments, the operation of the lamp did not cause an observable increase in the temperature of the airflow.

To prevent direct Far-UVC irradiation on the pre-mixing section and facilitate the installation of subsequent partitions for redirecting airflow, the rectangular lamp module was installed outside the disinfection box chamber at position (X=175 mm, Y=300 mm, Z=75 mm). The box chamber featured an opening measuring 50 mm×70 mm, which perfectly aligned with the illuminating window of the lamp module, enabling UV irradiation to enter the box chamber. This configuration called ITE system. In another system, which was equipped with a tubular lamp, a Far-UVC tube, with a diameter of 30 mm and a height of 120 mm, was centrally placed at the position (X=250 mm, Y=150 mm). A foundation with a 1.5 cm height was employed to secure the tube. This configuration called EME system.

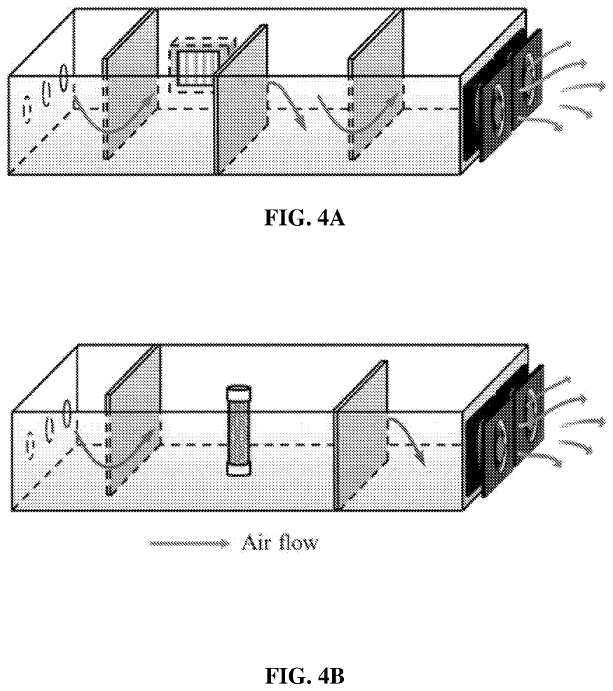

Referring to FIGS. 4A-4B, Additional acrylic plates could be installed inside the box chamber. These partitions were strategically positioned to guide contaminated air to the area with high-intensity UV irradiation. In the EME system, two additional acrylic plates were mounted at X=250 mm and X=400 mm. The plate at X=250 mm was designed to guide the contaminated air to the area with high UV irradiance, while another plate at X=400 mm was added to further increase exposure time by creating additional air resistance. In the ITE system, only one additional plate was mounted at X=400 mm. This additional plate was also intended redirect the airflow to high intensity zone. The airflow blockage was further adjusted by the plate length of the partition. In the following examples, two plate lengths were tested: 200 mm and 250 mm.

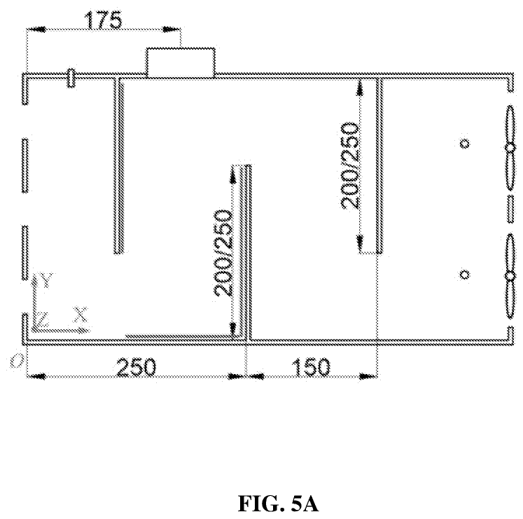

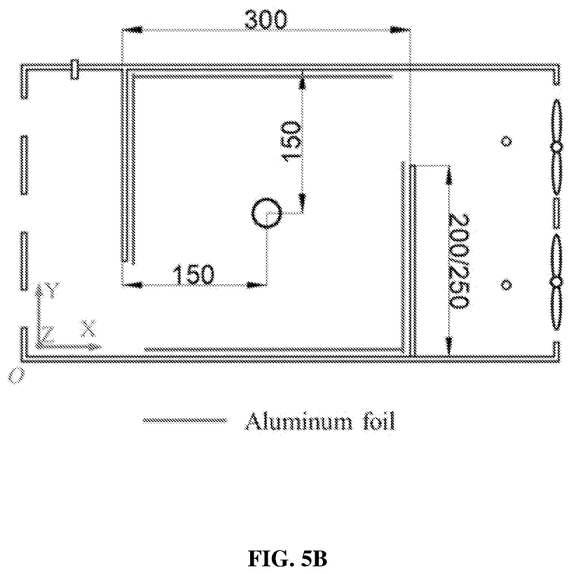

The second enhancement involved covering partial internal walls and partition walls with UV reflection material to enhance inter-wall reflection and further increase the UV irradiance. Turning to FIGS. 5A-5B, the internal walls and partition walls surrounding the Far-UVC lamps were covered with Al foil to enhance UV reflection, resulting in a further increase in the UV irradiance. In the EME system, the partition at X=400 mm was not covered with Al foil since the UV light cannot penetrate the partition at X=250 mm to reach the plate.

### Example 3—Procedure

To prevent external environmental interference, the experiments were conducted in an air-tight room with a leakage of less than 0.1 ACH (air changes per hour). The air in the room was approximately maintained at 20° C. and relative humidity 58% during the experiments. Before each experiment, the tested disinfection systems were sterilized with 70% ethanol and the room was pre-disinfected by a 36 W 254 nm UV lamp for 15 minutes. Once the pre-disinfection was finished, the O₃ concentration reduced rapidly in 2 minutes and could not be detected by the O₃ monitor. Subsequently, the suspension of pathogens was nebulized and injected into the system by a 6-jet nebulizer (CH Technologies, USA) with an air pressure of 20 psi (137.9 kPa) provided by an air compressor. To minimize the dispersion of microbes by the expelled air, an indoor air purifier was positioned near the outlet of the box chamber to collect the expelled microorganisms. Ten minutes was waited to ensure that the bioaerosol concentration inside the system reached a stable level. Air samples were then taken to investigate the concentration level of the microorganisms in the box chamber. A liquid impinger (Biosampler, SKC, USA) filled with 20 mL of 0.9% saline was connected to a vacuum pump with flow rate of 12.5 L/min for air sampling. A flexible tube was connected from the impinger sampling opening to point c₁. In addition, a 3 cm long tubing was attached to the interior part of c₁ and extended into the box chamber, allowing air to be sampled from the mainstream. After sampling at c₁ for 2 minutes, the liquid impinger was connected to point c₂ and the results were finally combined to determine the microbial concentration under UV-off conditions. The sampling flow rate was kept at 12.5 L/min by a vacuum pump. Next, the Far-UVC lamp was turned on and the above procedures were repeated to investigate the disinfection performance of the Far-UVC treatment.

After air sampling, a series of 10-fold dilutions were performed, followed by plate cultivation. For airborne bacteria, 50 μL of the diluted sample was spread uniformly on a solid NA plate, which was then incubated at 37° C. for 24 hours. The resulting colony-forming units (CFU) on the plates were countered for further study after incubation. For virus, 50 μL of diluted sample was mixed with 3 mL of melted 0.7% TSA and 50 μL of log-phase E. Coli. The mixture was then vortexed and evenly dispensed onto a 1.5% solid TSA plate. After 24 hours incubation at 37° C., the PFU on the plates were enumerated.

The inactivation efficacies against the three microbes of two baseline systems were initially tested individually to establish a baseline point. Subsequently, the enhancement measures were sequentially implemented for further investigation. For each experiment, the air sampling was only taken by one time. All experiments were repeated at least three times. In addition, bioaerosol survival can be affected by a number of environmental factors. To account for the natural loss and potential injury caused by nebulization and/or collection by impinger, control experiments were performed without Far-UVC treatment. These control experiments served as a comparison to evaluate the effectiveness of Far-UVC treatment in reducing microbial concentration.

### Example 4—UV Irradiance Distribution of the Portable and Filter-Free Single-Pass Disinfection Systems

A high-resolution fiber-optic spectrometer (ULS3648, Avantes, Netherlands) was used to measure the spatial Far-UVC irradiance. Since the optical sensor of the spectrometer only measures planar unidirectional irradiance, UV irradiance was measured from six orthogonal directions at each measuring point and subsequently combined to determine the total UV irradiation received by the microbes at that specific point. The volume-average UV irradiance was determined by averaging all the measured values in the irradiated area.

Since Far-UVC light cannot penetrate through the acrylic plates, the UV irradiance within the pre-mixing section from X=0 to X=100 was assumed to be negligible. The UV irradiance distributions were expected to be symmetrical about the Z=75 mm plane, due to the balanced positioning of the UV sources within the box chamber. Therefore, the UV irradiance distribution in the box chamber was measured at Z=25 mm and Z=75 mm to calculate the volume-average UV irradiance. Besides, due to the symmetrical layout of the ITE system, the irradiance measurement was further simplified. The interior zone from X=100 mm to X=550 mm was divided into four quadrants by the UV tube, located at the point (X=250 mm, Y=150 mm). The irradiance distribution in the first quadrant was measured first, and subsequently used to determine the spatial UV irradiance in other quadrants. Since the optical sensor of the spectrometer only measures planar unidirectional irradiance, UV irradiances were measured from six directions at each measuring point and then summed to account for the total irradiance received by the microbes at that point^{[24]}. The volume-average UV intensity was determined by the averaging all the measured values.

Because of the symmetrical layout of the system and the UV sources, the intensity distribution above the mid-plane (Z=75) was assumed to be mirrored to the lower counterpart. In this regard, the intensity distribution in the plane of Z=125 was assumed equal to that in the plane of Z=25. Also, the interior zone from X=100 to X=550 was divided into four quadrants by the UV tube, located at point (X=250, Y=150). The intensity distribution in the first quadrant was measured first, and subsequently used to determine the distribution in the other three quadrants.

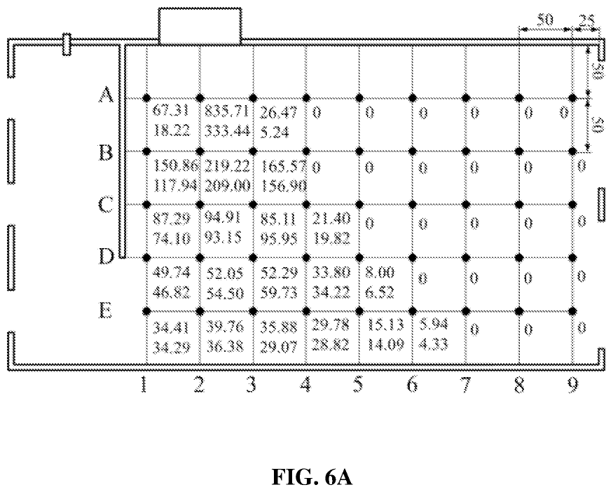

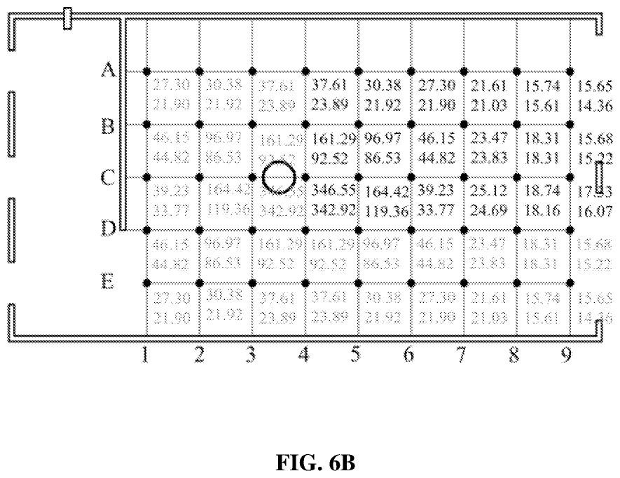

The detailed measurements of UV irradiance distributions in the baseline EME and ITE systems were presented in FIGS. 6A-6B. In the EME system, due to the small sizes of the illumination surface and limited beam angle of the rectangular module, only approximately half of the box chamber volume received UV irradiation. The volume-average UV irradiance of the entire box chamber in the baseline EME system was determined to be 37.4 μW/cm². In contrast, the baseline ITE system could uniformly irradiate the entire box chamber and had a significantly expanded internal UV-irradiated area with an average irradiance of 56.0 μW/cm².

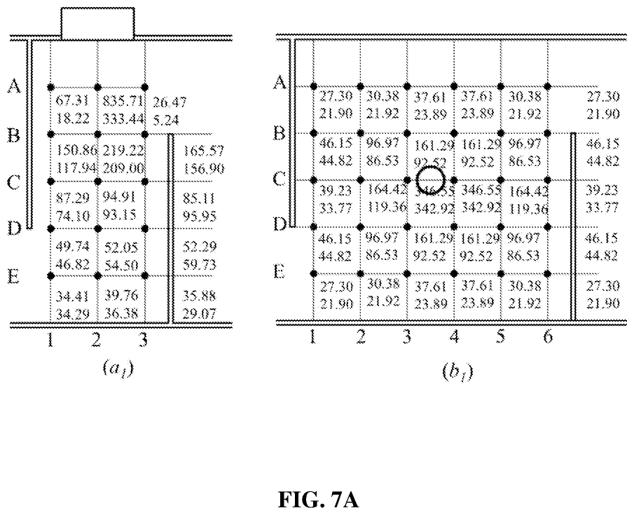

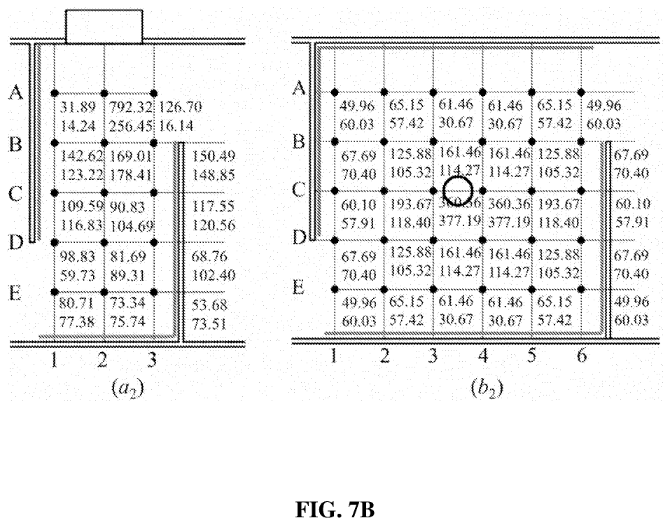

By inserting additional partitions, the UV treatment section was partitioned into several semi-enclosed areas, resulting in a modification of the original airflow. The purpose of these partitions was to redirect the airflow towards areas with high-intensity UV irradiation. rather than areas with relatively low UV irradiance. As shown in FIGS. 7A-7B, taking the partitioned EME system (FIG. 7A, a₁) as an example, the airflow was redirected to point A2, where the UV irradiance was measured over 300 μW/cm². In this irradiated area, the volume-average UV irradiance for the partitioned EME system was 105.0 μW/cm². Similarly, for the ITE system (FIG. 7A, b₁), the volume-average UV irradiance was 74.7 μW/cm² in the irradiated area. The spatial UV irradiance was expected to remain consistent when the partitions were extended to 250 mm.

Referring to FIG. 7B, when Al foil coating, the average irradiance in the UV irradiated area increased to 117.8 μW/cm² and 100.8 μW/cm² respectively for EME system (a₂) and ITE system (b₂), respectively. The enhancement ratio for the EME and ITE systems were 12.2% and 34.9% respectively. The relatively low enhancement for the EME system could be attributed to the arrangement of the partitions and the lamp. Since the partitions in the EME system were aligned parallel to the UV light emitted from the external lamp, the partition surfaces near the UV lamp received minimal direct irradiation. This configuration resulted in limited reflected UV light from the partitions. As shown in a₁. a₂, the measured UV irradiance at points A1, B1, and C1 increased minor when the A1 foil was applied. However, the aluminum foil on the wall opposite the lamp reflected UV light, significantly increasing the irradiance near the reflective surface. For example, the UV irradiance measured at all points on row E increased by at least 50% after applying the aluminum foil.

### Example 5—Flow Rate of the Portable and Filter-Free Single-Pass Disinfection Systems

The flow rate of the tested embodiment (with or without internal partitions) was measured by a vane anemometer (Model 417, Testo) near the exit of the system. The accompanying fitting of the anemometer allowed for the collection of the total exhausted airflow from the system, which then passed through the anemometer for measurement. The measurement for each configuration of the system lasted for 90 s. The measured flow rates were recorded every 30 seconds and finally averaged.

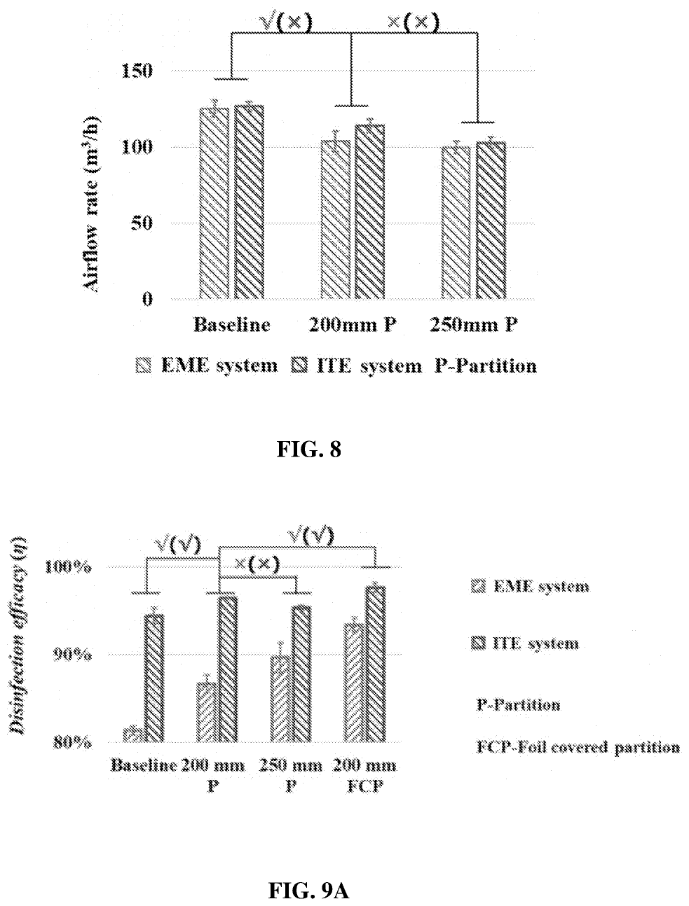

The airflow rates of the systems with various partition configurations were illustrated in FIG. 8. Based on these measurements, the Reynolds numbers of the airflow within the systems ranged from 8842 to 13379, indicating turbulent airflow. The baseline EME and ITE systems performed comparable airflow rates (125.3 m³/hr vs. 126.7 m³/hr), which indicated that the air resistance caused by the internal UV tube was insignificant.

By installing additional 200 mm long partitions (200 mm P group), the airflow rates of the EME system decreased by 17.2%, while the airflow rates of the ITE system showed a modest but statistically insignificant decrease of less than 10%. The difference in flowrate reduction indicated the effect of the second partition. Extending the length of partitions to 250 mm (250 mm P group) resulted in only a slight and statistically insignificant decrease in airflow rates compared with 200 mm long partitions (decrease ratios of 3.9% and 9.8% respectively).

Moreover, the airflow rate of the tested embodiment was mainly controlled by input current of the axial fans and the air resistance drag resulting from the number of partitions and the extend of the airflow blockage. Two input current settings were tested. The rated current of each fan was 1.6 A (3.2 A for two fans), while the minimum current required for stable fan operation is 0.9 A (1.8 A for two fans). Taking the ITE system as an example, the airflow rates were measured with different currents and partition plate lengths, and the results were listed in Table 1. The flow rates of the tested embodiment were reduced due to the 200 mm partitions. By extending the partitions to 250 mm, the flow rates were further reduced.

TABLE 1
flow rates of the ITE system with different
fan currents, partitions. (Unit: m³/h)

| | | 200 mm | |
|---|---|---|---|
| Fan current | No partition | partitions | 250 mm partitions |
| Minimum Current | 95.4 | 88.7 | 83.7 |
| Rated Current | 129.5 | 117.6 | 107.6 |

### Example 6—Microbial Inactivation Efficacy

The disinfection efficacy of the system was calculated by Eq (1).

$$C 2 / C 1 = 1 - \frac{( η + {η}^{′} )}{100 \%} \qquad ( 1 )$$

where C₁ (/m³) and C₂ (/m³) are the concentration of the airborne microorganisms during the two sampling periods, and the ratio of these concentrations indicates the survival fraction of the microbes that have been treated by the system.

As the durations for two samplings were the same, the concentration terms in the equation were substituted by the CFUs or PFUs counted from the agar plates used to culture the microorganisms collected from the sampled air, both with and without Far-UVC treatment. η′ is the concentration reduction between two sampling periods caused by other natural or environmental factors. During the control experiments, when UV was off, η′ can be calculated as:

$${η}^{′} = 1 - {C}_{2 , off} / {C}_{1 , off} \times 100 \% \qquad ( 2 )$$

where C_{2,off} and C_{1,off} are the concentration of the airborne microorganisms in system with Far-UVC off. A two-tailed paired t-test was applied to determine the significant difference between the disinfection efficacies obtained from different scenarios. The statistical significance was demonstrated by the p-value<0.05.

Based on the control experiments, η′ was measured as 0.57%±0.04. This low value indicated that the effects of non-UV factors on the experimental results were negligible.

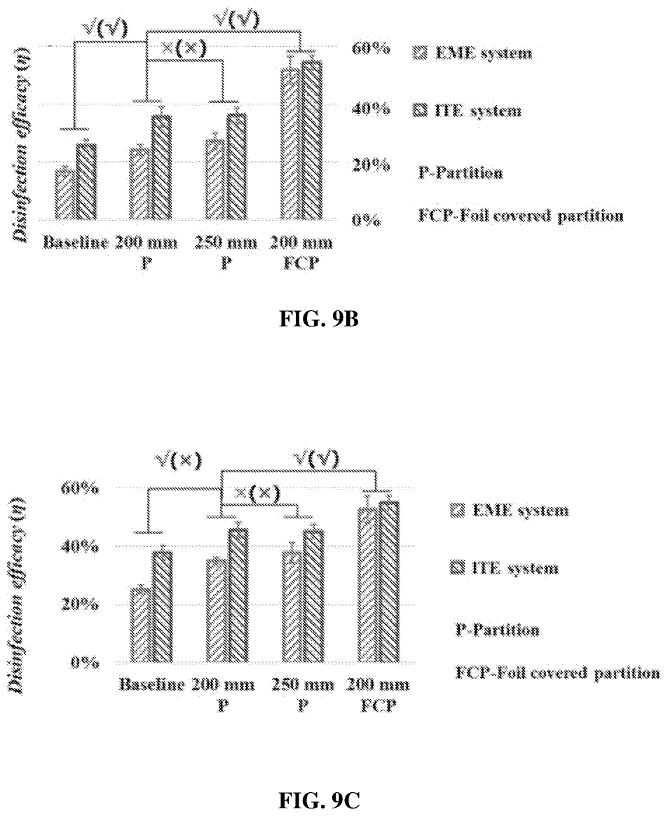

FIGS. 9A-9C illustrated the inactivation efficacies of two baseline systems and the effect of modifications. The inactivation efficacies were obtained by averaging at least three sets of experimental data. In the baseline EME system, the inactivated efficacies against S. enterica, S. epidermidis, and MS2 were 81.4%, 16.9%, and 25.1% respectively. On the other hand, the baseline ITE system presented higher efficacies against the same microorganisms, with values of 94.4%, 25.8%, and 37.8%. The differences in inactivation efficacies between the baseline EME and ITE systems were 16.0%, 53.1%, and 50.3%, respectively. The difference may be attributed to the wider irradiation area and higher power output of the UV lamp in the ITE system.

The relationship between the output of the UV lamp and the inactivation efficacy of the system follows the Bunsen-Roscoe reciprocity law, as described by the equation:

$$1 - η = {e}^{- Z * D} = {e}^{- Z * I * t} \qquad ( 3 )$$

where Z (m²/J) is the Far-UVC susceptibility constant of the microbe; D (J/m²) is the UV irradiation dose received by the microbe, which is a product of the average UV irradiance (I, W/m²) and the UV exposure time (t, s).

The estimated inactivation efficacies of the baseline ITE system, equipped with a 15 W UV tube, against the three tested microbes are 91.9%, 22.9%, and 33.8%. These values are 12.9%, 35.7%, and 34.7% higher than the inactivation efficacies of the baseline EME system against S. enterica, S. epidermidis, and MS2, respectively. This indicated the advantageous effect of the tubular geometry of the UV source in the baseline systems.

Moreover, the installation of additional 200 mm long partitions resulted in improved inactivation efficacies compared to the baseline systems. The EME system presented a minor improvement of 6.5% in the inactivation efficacy against S. enterica, while the ITE system showed a 2.2% improvement. However, the survival fraction (1-n) of S. enterica was significantly reduced. In the EME system, the survival fraction decreased from 18.6% to 13.3% corresponding to a decrease ratio of 28.4%. Similarly, in the ITE system, the survival fraction decreased from 5.6% to 3.5%, with a decrease ratio of 36.6%. The inactivation efficacies against S. epidermidis and MS2 showed impressive improvements: 43.5% and 38.7% for the EME system and 37.7% and 20.6% for the ITE system. However, the improvement of the ITE system in inactivating airborne MS2 was statistically insignificant, indicated by p-value=0.13. When the length of partitions was extended to 250 mm, the inactivation efficacies of the systems showed a modest and statistically insignificant improvement compared to the efficacies of the systems with 200 mm long partitions.

Compared with partition extension, the application of Al foil resulted in a significant improvement of the inactivation efficacy. For 200 mm partitions equipped EME system, the Al foil coating enhanced the inactivation efficacies against S. enterica, S. epidermidis and MS2 to 93.4%, 51.8%, and 52.5%. The enhancement ratios were 7.7%, 113.9%, and 50.7% respectively. For the ITE system, the inactivation efficacies against the three microbes were improved to 97.6%, 54.4%, and 55.1%, with enhancement ratios of 1.2%, 53.0%, and 21.0% respectively. Decreasing the power of the UV tube to 15 W, the inactivation efficacies of the modified ITE system against the three microbes were estimated as 96.1%, 49.5%, and 50.2% based on Eq (3).

### Example 7—Air Disinfection Performance of the Disinfection System

In one previous study, the air disinfection efficacy of an upper-room Far-UVC fixture was tested in a room with 11.9 m³ volume under well-mixed air conditions¹⁰. In this example, the EME system and ITE system were hypothetically used in the same room, assuming they also operated under well-mixed conditions to compare the disinfection performance between the proposed portable air disinfection systems and a wall-mounted upper-room Far-UVC fixture.

The reduction in airborne microbial concentration in a well-mixed condition, attributable to the disinfection systems, could be determined based on mass balance:

$$ln ( \frac{ct}{c 0} ) = \frac{- \text{?} Q}{v} t = - k t \qquad ( 4 )$$

$$\text{?} \text{indicates text missing or illegible when filed}$$

where Q is the air flow rate treated by the system (m³/hr); C is the indoor bioaerosols concentration (/m³) while C₀ is the initial concentration (/m³); t is the operating duration of the system (hr); V is the volume of the room (m³). Meanwhile, k (hr-1) is the bioaerosols concentration decay rate caused by UV inactivation. It is equivalent to the concept of ACH when it is expressed in units of hr-1.

In this semi-hypothetical scenario for indoor air disinfection, the EME or ITE system, modified with 200 mm partitions covered in aluminum foil, was applied. The equivalent ACH provided by the system could be estimated using Eq (4) and compared with the results from the previous study on upper-room Far-UVC, as presented in Table 2. The power consumptions of the UV sources used in this calculation were all set at 15 W, which was the power consumption of the Far-UVC lamp used in the upper-room experiments. Thus, the inactivation efficacies of the modified ITE system with 20 W power Far-UVC tube were adjusted to those with 15 W power based on Eq (3).

TABLE 2
The equivalent ACH (hr⁻¹) of microbial inactivation
by the disinfection systems and upper-room Far-UVC¹⁰

| | S. enterica | S. epidermidis | MS2 |
|---|---|---|---|
| EME system | 8.14 | 4.52 | 4.58 |
| ITE system | 9.21 | 4.74 | 4.80 |
| Upper-room Far-UVC | 28.44 | 3.67 | |

In Table 2, compared to the upper-room Far-UVC, the EME and ITE disinfection systems demonstrated higher equivalent ACH of removing S. epidermidis by 23.0% to 29.2%, respectively. However, it should note that these disinfection systems showed significantly slower inactivation against S. enterica, nearly 20 hr⁻¹ less, compared to the upper-room installation. This suggested that the upper-room Far-UVC installation holds an advantage in disinfecting microorganisms that are highly susceptible to Far-UVC light, while the disinfection systems performed better in inactivating Far-UVC-resistant microorganisms due to their ability to provide localized, confined high UV irradiance for air disinfection. In practical applications, the air contains a wide variety of microorganisms that need to be inactivated, not just a single type. Since the disinfection process duration is often determined by the presence of UV-resistant microorganisms, the high inactivation efficacy of these systems against such microorganisms is clearly advantageous compared to upper-room UVGI.

In summary, the present invention provides single-pass air disinfection systems utilizing Far-UVC irradiation. The systems' inactivation efficacy against bacteria, including S. enterica and S. epidermidis, as well as the bacteriophage MS2, has been evaluated at high flow rates exceeding 100 m³/hr. The significantly uneven distribution of UV irradiance produced by the rectangular lamp is a key factor contributing to the inferior disinfection performance of the baseline EME system compared to the ITE system.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated.

### Definition

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. Patent law; e.g., they allow for elements not explicitly recited, but exclude elements that are found in the prior art or that affect a basic or novel characteristic of the present invention.

Furthermore, throughout the specification and claims, unless the context requires otherwise, the word “include” or variations such as “includes” or “including”, will be understood to imply the inclusion of a stated integer or group of integers but not the exclusion of any other integer or group of integers.

As used herein and not otherwise defined, the terms “substantially,” “substantial,” “approximately” and “about” are used to describe and account for small variations. When used in conjunction with an event or circumstance, the terms can encompass instances in which the event or circumstance occurs precisely as well as instances in which the event or circumstance occurs to a close approximation. For example, when used in conjunction with a numerical value, the terms can encompass a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

References in the specification to “one embodiment”, “an embodiment”, “an example embodiment”, etc., indicate that the embodiment described can include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

In the methods of preparation described herein, the steps can be carried out in any order without departing from the principles of the invention, except when a temporal or operational sequence is explicitly recited. Recitation in a claim to the effect that first a step is performed, and then several other steps are subsequently performed, shall be taken to mean that the first step is performed before any of the other steps, but the other steps can be performed in any suitable sequence, unless a sequence is further recited within the other steps. For example, claim elements that recite “Step A, Step B, Step C, Step D, and Step E” shall be construed to mean step A is carried out first, step E is carried out last, and steps B, C, and D can be carried out in any sequence between steps A and E, and that the sequence still falls within the literal scope of the claimed process. A given step or sub-set of steps can also be repeated. Furthermore, specified steps can be carried out concurrently unless explicit claim language recites that they be carried out separately.

The term “single-pass” refers to the process where the airflow or bioaerosols pass through the disinfection system only once to achieve the desired level of disinfection. This means that the air only needs to go through the system a single time to be effectively disinfected, without requiring multiple passes or additional filtration stages. This design aims to enhance disinfection efficiency while simplifying the system and reducing operational costs.

The term “pathogen” refers to a biological agent that causes disease or illness to its host. Pathogens include a variety of microorganisms such as bacteria, viruses, fungi, and parasites. They can invade and multiply within the host's body, often triggering an immune response and leading to various symptoms or health issues.

The term “contaminated airflow” refers to a stream of air that contains pollutants, harmful microorganisms, or other undesirable substances. These contaminants can include dust, bacteria, viruses, mold spores, chemical vapors, and other airborne particles that can pose health risks or negatively impact indoor air quality. Managing contaminated airflow is crucial in environments such as hospitals, laboratories, industrial settings, and residential areas to ensure a safe and healthy atmosphere.

The term “bioaerosols” refer to tiny biological particles suspended in the air, including bacteria, viruses, fungal spores, pollen, animal dander, and other microorganisms or their metabolic products. These particles typically originate from natural environments like soil, plants, and water bodies, as well as human activities such as agriculture, industry, and healthcare facilities.

High airflow rate” refers to the volume of air that moves through a given space or system in a specific period, typically measured in units such as cubic feet per minute (CFM) or liters per second (L/s). A high airflow rate indicates that a large amount of air is being circulated or exchanged quickly. For instance, a high airflow rate can be considered as at least 100 cubic meters per hour (m³/h).

Other definitions for selected terms used herein may be found within the detailed description of the present invention and apply throughout. Unless otherwise defined, all other technical terms used herein have the same meaning as commonly understood to one of ordinary skill in the art to which the present invention belongs.

### INDUSTRIAL APPLICABILITY

The present invention enhances the efficacy of single-pass air disinfection in portable disinfection systems by employing various Far-UVC light sources. It has low initial cost, low operation cost, and a long lifetime. The designed system enclosed the UVGI unit inside the air chamber to avoid direct UV exposure to occupants. Thus, the system can be used even when there are occupants in the room. Meanwhile, by the modifications mentioned above, the system gives very good disinfection efficiencies even with high airflow rates. Hence, the system is expected to provide continuous and reliable indoor air disinfection for indoor occupants.

The enhancements made to the portable single-pass disinfection systems of the present invention focus on improving disinfection performance. These improvements build upon two original concepts: (i) redirecting contaminated airflow and (ii) enhancing internal UV irradiance.

The system's portability makes it versatile and applicable in many different scenarios. The system can not only be used for room disinfection but also for public transportation. It can be used as a standalone air disinfection system or be combined with existing technology for further reduction of airborne pathogen concentration.

### REFERENCES: THE DISCLOSURES OF THE FOLLOWING REFERENCES ARE INCORPORATED BY REFERENCE

1. Nazarenko. Y. (2020). Air filtration and SARS-CoV-2. Epidemiology and health, 42.
2. Lu, Z., Lu, W. Z., Zhang, J. L., & Sun. D. X. (2009). Microorganisms and particles in AHU systems: Measurement and analysis. Building and Environment, 44 (4). 694-698.
3. Lee, L. D., Delclos, G., Berkheiser, M. L., Barakat, M. T., & Jensen, P. A. (2022). Evaluation of multiple fixed in-room air cleaners with ultraviolet germicidal irradiation, in high-occupancy areas of selected commercial indoor environments. Journal of Occupational and Environmental Hygiene. 19 (1), 67-77.
4. Burdack-Freitag, A., Buschhaus, M., Grün, G., Hofbauer, W. K., Johann, S., Nagele-Renzl, A. M., . . . & Scherer, C. R. (2022). Particulate Matter versus Airborne Viruses—Distinctive Differences between Filtering and Inactivating Air Cleaning Technologies. Atmosphere, 13 (10), 1575.
5. Burdack-Freitag A, Buschhaus M, Grün G, et al. Particulate matter versus airborne viruses-distinctive differences between filtering and inactivating air cleaning technologies. Atmosphere. 2022; 13:1575.
6. Snelling. W. J., Afkhami, A., Turkington, H. L., Carlisle, C., Cosby, S. L., Hamilton, J. W., . . . & Dunlop, P. S. (2022). Efficacy of single pass UVC air treatment for the inactivation of coronavirus, MS2 coliphage and Staphylococcus aureus bioaerosols. Journal of Aerosol Science, 164, 106003.
7. Nunayon, S. S., Zhang, H. H., & Lai, A. C. (2020). A novel upper-room UVC-LED irradiation system for disinfection of indoor bioaerosols under different operating and airflow conditions. Journal of hazardous materials, 396, 122715.
8. Nunayon, S. S., Zhang, H., & Lai, A. C. (2020). Comparison of disinfection performance of UVC-LED and conventional upper-room UVGI systems. Indoor Air, 30 (1), 180-191.
9. Kim, D. K., & Kang, D. H. (2018). UVC LED irradiation effectively inactivates aerosolized viruses, bacteria, and fungi in a chamber-type air disinfection system. Applied and environmental microbiology, 84 (17), c00944-18.
10. Nunayon, S. S., Zhang, H. H., Chan, V., Kong, R. Y., & Lai, A. C. (2022). Study of synergistic disinfection by UVC and positive/negative air ions for aerosolized Escherichia coli, Salmonella typhimurium, and Staphylococcus epidermidis in ventilation duct flow. Indoor air, 32 (1), e12957.

What is claimed is:

1. A portable and filter-free single-pass air disinfection system for disinfection of contaminated airflow or bioaerosols, wherein the portable and filter-free single-pass air disinfection system comprises:
a disinfection chamber comprising:
a premixing zone, wherein the premixing zone comprises at least one air inlet for introducing the contaminated airflow or the bioaerosols into the disinfection chamber;
a treatment zone, wherein the treatment zone comprises an ultraviolet (UV) irradiation source configured to irradiate the contaminated airflow or the bioaerosols within the disinfection chamber; and
a sampling zone, wherein the sampling zone comprises a liquid impinger connected to a vacuum pump for air sampling, and at least one vent outlet for cleaned air; and
one or more exhaust fans equipped on one side of the disinfection chamber to facilitate airflow through the disinfection chamber,
wherein the premixing zone, the treatment zone and the sampling zone are interconnected,
wherein at least one partition is positioned between the premixing zone and the treatment zone.

2. The portable and filter-free single-pass air disinfection system of claim 1, wherein the UV irradiation source is a Far-UVC source emits light at a wavelength of approximately 222 nm, the Far-UVC source comprises a tubular lamp or a rectangular lamp.

3. The portable and filter-free single-pass air disinfection system of claim 2, wherein the rectangular lamp is positioned externally to the disinfection chamber.

4. The portable and filter-free single-pass air disinfection system of claim 2, wherein the tubular lamp is positioned internally within the treatment part.

5. The portable and filter-free single-pass air disinfection system of claim 1, wherein the at least one partition re-directs the contaminated airflow or the bioaerosols to extend an airflow pathway to a region with high UV irradiation.

6. The portable and filter-free single-pass air disinfection system of claim 1, wherein the at least one partition and internal walls of the disinfection chamber are covered with reflected materials with a reflectivity of at least 90% to enhance UV reflectance, the reflected materials comprise aluminum foil.

7. The portable and filter-free single-pass air disinfection system of claim 1, wherein the portable and filter-free single-pass air disinfection system has an airflow rate of at least 100 m³/h.

8. The portable and filter-free single-pass air disinfection system of claim 7, wherein the portable and filter-free single-pass air disinfection system inactivates pathogens in the contaminated airflow or bioaerosols.

9. The portable and filter-free single-pass air disinfection system of claim 7, wherein the portable and filter-free single-pass air disinfection system achieves an inactivation rate of at least 80% for Salmonella enterica, at least 15% for Staphylococcus epidermidis, and at least 20% for MS2 bacteriophage.

10. The portable and filter-free single-pass air disinfection system of claim 1, further comprising an input terminal connected to a current input device for controlling current of the one or more exhaust fans.

11. The portable and filter-free single-pass air disinfection system of claim 1, wherein the disinfection chamber is made of at least one acrylic plate with a thickness of 1 mm to 10 mm.

12. A method for disinfection of contaminated airflow or bioaerosols, comprising the steps of:
preparing a filter-free disinfection chamber having a premixing zone, a treatment zone and a sampling zone, wherein at least one partition is inserted between the premixing zone and the treatment zone;
introducing the contaminated airflow or the bioaerosols into the premixing zone;
directing the contaminated airflow or the bioaerosols towards the treatment zone, wherein the treatment zone comprises an ultraviolet (UV) irradiation source configured to irradiate the contaminated airflow or the bioaerosols within the disinfection chamber, wherein the UV irradiation source comprises Far-UVC source emits light at a wavelength of approximately 222 nm; and
collecting and analyzing irradiated air samples to determine microbial concentration.

13. The method of claim 12, wherein one or more exhaust fans are equipped on one side of the disinfection chamber to facilitate airflow through the disinfection chamber.

14. The method of claim 12, the Far-UVC source comprises a tubular lamp or a rectangular lamp.

15. The method of claim 14, wherein the rectangular lamp is positioned externally to the disinfection chamber.

16. The method of claim 14, wherein the tubular lamp is positioned internally within the treatment zone.

17. The method of claim 12, further comprising step of covering the at least one partition and internal walls of the disinfection chamber with reflected materials with a reflectivity of at least 90% to enhance UV reflectance.

18. The method of claim 12, further comprising step of pre-disinfecting the disinfection chamber with an UV lamp before introduction of the contaminated airflow or the bioacrosols.

19. The method of claim 12, further comprising step of installing a safety mechanism to automatically shut off the UV irradiation source if the disinfection chamber is opened during operation.
